(19) United States
(12) Patent Application Publication
(10) Pub. No.: US 2013/0266532 A1
(43) Pub. Date: Oct. 10, 2013

(54) ORGANIC FERTILIZER COMPOSITION, AND METHOD FOR PREPARING SAME

(71) Applicant: Cheon Gun Kim, Gyeonggi-do (KR)

(72) Inventor: Cheon Gun Kim, Gyeonggi-do (KR)

(21) Appl. No.: 13/808,587

(22) PCT Filed: Jul. 5, 2011
(86) PCT No.: PCT/KR2011/004911

§ 371 Date: Jun. 24, 2013

(30) Foreign Application Priority Data
Jul. 5, 2010 (KR) 10-2010-0064403

(51) Int. Cl. C05F 17/00; C02F 1/00; A01N 65/08; A61K 36/82; A61L 9/013

(52) U.S. Cl. CPC C05F 17/0027; A61K 36/82; A61L 9/013; A01N 65/08; C02F 1/00
USPC 424/76.1; 71/8; 424/115; 252/180

(57) ABSTRACT

An organic fertilizer composition which can be used as organic compost, and a method for preparing the composition. The organic fertilizer composition can be used as compost alone or together with well-known compost. The organic fertilizer composition is prepared by: a culturing step of mixing either grape sugar, starch syrup, brown sugar, or molasses, or a mixture thereof with plant leaves selected from among those of grass, vegetables, or herbs, or a mixture thereof, adding salt to the resultant mixture to obtain a first fermented mixture, and culturing the first fermented mixture at a temperature of 20 to 60° C. for 24 hours to 1 year; a propagation step of mixing together the first fermented mixture, water, and either grape sugar, starch syrup, brown sugar, or molasses, or a mixture thereof, adding salt to the resultant mixture to obtain a second fermented mixture, and propagating the second fermented mixture at a temperature of 20 to 40° C.; and a mass propagation step of mixing together the second fermented mixture, water, and either grape sugar, starch syrup, brown sugar, or molasses, or a mixture thereof, adding salt to the resultant mixture to obtain a third fermented mixture, and mass-propagating the third fermented mixture at a temperature of 20 to 40° C. for 1 to 12 hours, wherein said mass propagation step is optional. The organic fertilizer composition can be variously used as animal feed, a soil conditioner, a water quality improvement agent, livestock feed, a malodor scavenger, etc.

### TECHNICAL FIELD

The present disclosure relates to an organic fertilizer composition prepared by culturing and proliferating microorganisms naturally inhabiting the leaves of various plants such as grasses, vegetables, herbs, green tea, etc. and a method for preparing same. The organic fertilizer composition allows organic farming without resorting to organic synthetic agricultural chemicals or chemical fertilizers.

### BACKGROUND ART

In the 1960s and 1970s, the use of compost was not common owing to the yield-oriented agricultural policy. Recently, however, quality of agricultural products is valued highly and high-quality agricultural products produced through the so-called ecofriendly, low-chemical organic farming are sold at high costs.

Also, people's interest in ecofriendly products is increasing as well-being and sustainable consumption attract the public nationally and globally. Such interest is higher in agricultural products and many people take a lot of interest in ecofriendly agriculture using naturally materials only such organic matter, natural minerals, microorganisms, etc. without resorting to synthetic chemical substances such as chemical fertilizers, organic synthetic agricultural chemicals, livestock feed additives, or the like.

Meanwhile, as the global use of fertilizers increases in proportion to agricultural production, decreased sustainability of the agroecosystem and environmental pollutions such as pollution of soil and underground water are causing concerns. In addition, the increased use of fertilizers resulted in excessive accumulation of nutrients in soil. In particular, the soil of the cultivation sites has aggravated to such an extent that soil salinity is worried about owing to excessive accumulation of salts.

### DISCLOSURE

### Technical Problem

At present, agroforestry byproducts such as rice hulls, sawdust, rice straw, etc., livestock excretions, food processing byproducts, fish processing byproducts, wastes or the like are fermented for several days after control of water content for use as compost. However, in most of the existing compost fermenting methods, the raw materials are mixed and fermented in a fermenter for only about 2 weeks with forced air blowing and shaking. Accordingly, insufficiently fermented compost is obtained in many cases.

Meanwhile, although organic farming using fermented compost can solve the problems associated with use of chemicals, considerable time and cost are required and the crops do not grow well since they become vulnerable to diseases and insects. In addition, since the harvest tends to be poor, the cost of the agricultural products increases steeply.

The present disclosure is directed to providing an organic fertilizer composition and a method for preparing same. More specifically, it relates to an organic fertilizer composition prepared by culturing and proliferating various useful microorganisms naturally inhabiting various plants such as grasses, vegetables, herbs, green tea, etc., which can be treated onto soil to improve the soil to be suitable for growth of the crops and improve the quality of agri-livestock products and allows organic farming without resorting to chemical fertilizers or organic synthetic agricultural chemicals (growth regulators, herbicides, etc.), and a method for preparing same.

The present disclosure is also directed to providing a compost including only the organic fertilizer composition and to providing a compost prepared by fermenting livestock excretions, food waste, etc. using same. The disclosure is also directed to providing a soil conditioner, a water quality improving agent, a livestock feed additive and a stench eliminator including the organic fertilizer composition as an active ingredient. The organic fertilizer composition according to the present disclosure may be used either alone or as mixed with a known soil conditioner, water quality improving agent, livestock feed additive or stench eliminator.

### Technical Solution

In one general aspect, the present disclosure provides an organic fertilizer composition prepared through fermentation and aging using useful microorganisms naturally occurring in plants.

The organic fertilizer composition according to the present disclosure is prepared by culturing, proliferation and mass proliferation. Specifically, it is prepared by: culturing a first fermentation mixture including 100 parts by weight of leaves of one or more plant selected from a grass, a vegetable and a herb, 55-150 parts by weight of one or more sugar selected from glucose, starch syrup, brown sugar and molasses and 0.1-3 parts by weight of salt at 20-60° C. for 24 hours to 1 year; proliferating a second fermentation mixture including 100 parts by weight of the first fermentation mixture, 100 parts by weight of water, 2.5-10 parts by weight of one or more sugar selected from glucose, starch syrup, brown sugar and molasses and 0.1-3 parts by weight of salt at 20-40° C. for 30 minutes to 24 hours; and mass proliferating a third fermentation mixture including 100 parts by weight of the second fermentation mixture, 100 parts by weight of water, 2.5-10 parts by weight of one or more sugar selected from glucose, starch syrup, brown sugar and molasses and 0.1-3 parts by weight of salt at 20-40° C. for 1-12 hours.

Since the organic fertilizer composition prepared by the above method includes only ecofriendly natural materials such as organic matter and the microorganisms habituating the leaves of grasses, vegetables, herbs, etc., it may be used to improve soil quality and cultivate agricultural products free of ingredients harmful to the human body. Hereinafter, each step of the method for preparing the organic fertilizer composition according to the present disclosure will be described in detail.

1. Culturing

In the culturing step, a first fermentation mixture is prepared by mixing 100 parts by weight of leaves of one or more plant selected from a grass (or leaf), a vegetable and a herb with 55-150 parts by weight of one or more sugar selected from glucose, starch syrup, brown sugar and molasses and 0.1-3 parts by weight of salt. The first fermentation mixture is cultured at 20-60° C. for at least 12 hours, specifically for at least 24 hours, more specifically for 1 day to 1 year. The culturing may be carried out in a commonly used open-topped container or entirely-closed container. Alternatively, a microorganism fermentation tank as shown in FIG. 1 may be used. Any device or apparatus can be used without limitation as long as the above-described temperature and time conditions are satisfied.

The leafs may be one or more selected from mugwort, pumpkin leaf, perilla leaf, mulberry leaf, persimmon leaf, sweet potato leaf, corn leaf, green tea leaf, cabbage leaf, potato leaf, etc. Any annual or biennial herbaceous plant having a soft stem and lacking woody tissue may be used without limitation.

The vegetable or the herb is not particularly limited. The vegetable may be one or more selected from radish, Chinese cabbage, onion, sweet potato, potato, pumpkin, paprika, pimento, cucumber, etc. Although a fresh vegetable is preferred, a wilted vegetable may also be used. The herb may be one or more selected from licorice, Angelica gigas radix, corni fructus, lycii fructus, etc.

In the culturing step, the leaves of the plants in which naturally occurring microorganisms exist are mixed with the sugar and salt at the proportions described above to obtain the first fermentation mixture. The first fermentation mixture comprises a large quantity of microorganisms such as Bacillus, Pseudomonas, Trichoderma, Lactobacillus, Cellulomonas, Hyphomicrobiaceae and Erwinia as an active ingredient of the organic fertilizer composition according to the present disclosure. The kind or quantity (or concentration) of these microorganisms are not limited and any microorganism occurring under the above-described condition may be used. Accordingly, the scope of the present disclosure is not limited by a specific kind or concentration of the microorganisms.

2. Proliferation

In the proliferation step, a second fermentation mixture is prepared by mixing 100 parts by weight of the first fermentation mixture with 100 parts by weight of water, glucose, 2.5-10 parts by weight of one or more sugar selected from glucose, starch syrup, brown sugar and molasses and 0.1-3 parts by weight of salt. The second fermentation mixture is proliferated at 20-40° C. for at least 30 minutes, specifically for 1-24 hours.

3. Mass proliferation In the mass proliferation step, a third fermentation mixture is prepared by mixing 100 parts by weight of the second fermentation mixture with 100 parts by weight of water, 2.5-10 parts by weight of one or more sugar selected from glucose, starch syrup, brown sugar and molasses and 0.1-3 parts by weight of salt. The third fermentation mixture is mass proliferated at 20-40° C. for at least 1 hour, more specifically for 1-12 hours. In this step, the microorganisms proliferated in the previous step may be proliferated in large scale, thereby reducing production cost and extending applicability of the organic fertilizer composition. However, the mass proliferation step may be omitted depending on the required quantity of the microorganisms, the type of agricultural application, or the like.

The organic fertilizer composition prepared according to the present disclosure can be used in itself as a compost suitable for organic farming, particularly as a liquid fertilizer.

In another general aspect, the present disclosure provides a fertilizer suitable for organic farming prepared by mixing the organic fertilizer composition with livestock excretions or food waste and performing refermentation. The refermentation may be carried out at 10-45° C. for at least 1 minute, more specifically for 2-5 days. The livestock excretions may be from any livestock including cow, pig, horse, chicken, ostrich, duck, rabbit, etc. Specifically, the livestock may be those raised using a feed mixed with the organic fertilizer composition according to the present disclosure. By fermenting the waste, a useful compost or liquid fertilizer can be obtained since the salinity and offensive odor of the waste can be reduced. And, by using the liquid fertilizer obtained from refermentation of the livestock excretions, crops can be cultivated without using any agricultural chemical.

The refermentation may be carried out in a commonly used open-topped container or entirely-closed container. Alternatively, as shown in FIG. 2, it may be carried out in an apparatus wherein food waste or livestock excretions are fermented to prepare a compost, which is connected to a microorganism culture tank. Any device or apparatus can be used without limitation as long as the above-described temperature and time conditions are satisfied.

In another general aspect, the present disclosure provides a soil conditioner comprising the organic fertilizer composition. The soil conditioner may comprise only the organic fertilizer composition of the present disclosure or may be prepared by mixing livestock excretions or food waste with the organic fertilizer composition of the present disclosure and performing refermentation. The refermentation may be carried out at 20-45° C. for at least 1 minute, more specifically for 2-5 days. The soil conditioner may be mixed with the soil used to cultivate crops.

In another general aspect, the present disclosure provides a water quality improving agent comprising the organic fertilizer composition. The water quality improving agent comprising the organic fertilizer composition of the present disclosure may be sprayed onto river, sea or a water tank (e.g., for fish farming). For example, when it is sprayed on the sea area where layer or pearl oyster is grown, red tide, algal bloom or water fouling may be prevented. And, when it is added, for example, to a water tank, moss may be prevented and water quality may be improved by decomposing harmful substances such as chlorine. As a result, fish can grow well. Also, when the water quality improving agent is sprayed onto river, wastewater from plants may be purified by the fertilizer composition of the present disclosure. As a result, water quality of the river can be improved and the river ecosystem can be protected. The water quality improving agent which comprises the organic fertilizer composition according to the present disclosure in addition to various known water quality improving agents may provide better water quality-improving effect than the existing water quality improving agents.

In another general aspect, the present disclosure provides a livestock feed additive comprising the organic fertilizer composition. The livestock feed additive may comprise 0.1-3 parts by weight of the organic fertilizer composition of the present disclosure in addition to a known livestock feed and may significantly reduce stench of excretions when fed to cow, pig, chicken, duck, horse, etc. And, a good liquid fertilizer (manure or compost) may be obtained by fermenting livestock excretions with the composition of the present disclosure. Further, it may improve the health condition of livestock when it is fed to them. A feed which comprises the organic fertilizer composition according to the present disclosure as additive in addition to various known feeds promotes the growth of livestock and prevents environmental pollution associated with livestock raising.

In another general aspect, the present disclosure provides a stench eliminator comprising the organic fertilizer composition according to the present disclosure. The stench eliminator comprises the organic fertilizer composition according to the present disclosure as additive in addition to various known stench eliminators and provides better stench eliminating effect than the existing stench eliminators.

### Advantageous Effects

As described above, the organic fertilizer composition according to the present disclosure, which is prepared under good culturing and proliferation conditions, may be used in various industrial applications as fertilizer and soil conditioner and as additive of water quality improving agent, livestock feed and stench eliminator.

In particular, it is very useful in that, when used as compost for organic farming, it allows organic farming of crops without resorting to chemicals.

### DESCRIPTION OF DRAWINGS

FIG. 1 shows a sectional elevation and a plan view of a fermentation tank that can be used in the present disclosure.

FIG. 2 is a schematic view of an apparatus for preparing a compost from food waste or livestock excretions that can be used in the present disclosure.

FIG. 3 shows a result of cultivating red pepper in Test Example 2.

FIG. 4 and FIG. 5 shows a result of cultivating rice in Test Example 3.

FIG. 6 shows a result of water quality improvement in Test Example 7.

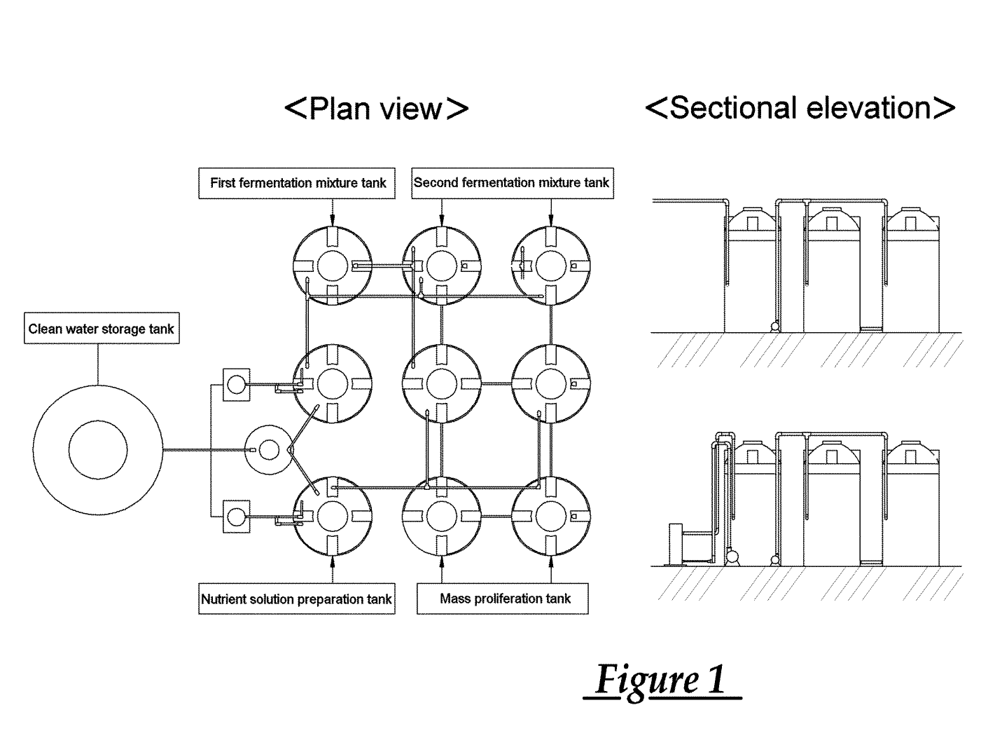

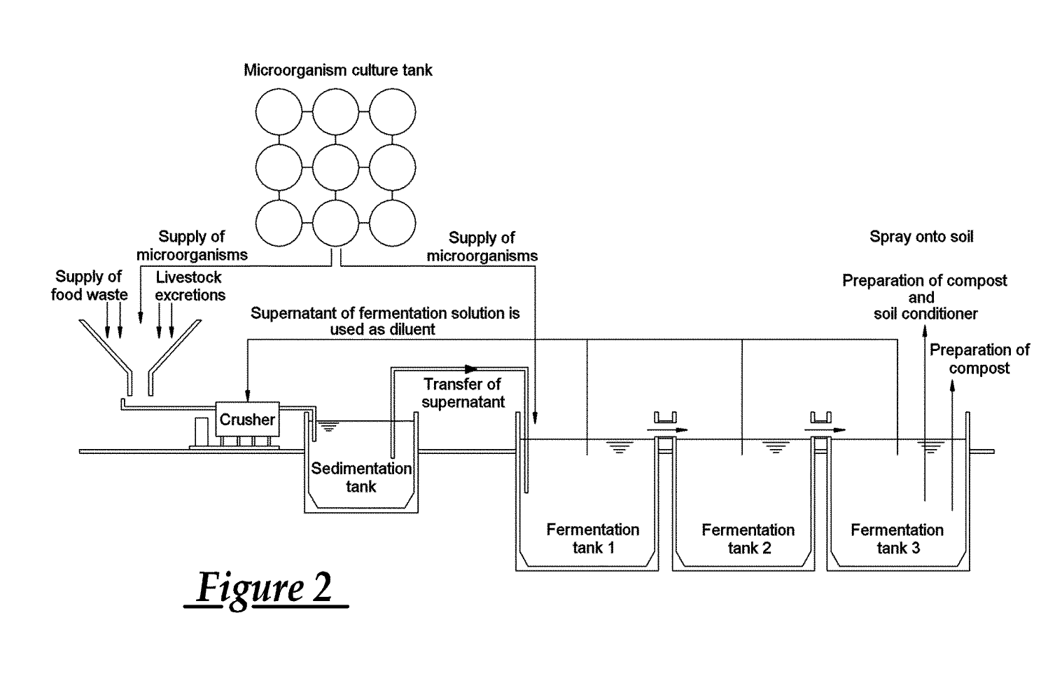

### MODE FOR INVENTION

### Example 1

### Preparation of First Fermentation Mixture (1)

100 parts by weight of the leaves of plantain, mugwort and green tea (1:2:6, based on weight) was mixed with 100 parts by weight of starch syrup and brown sugar (10:1) and 1 part by weight of salt. The mixture was cultured at 30° C. for 24 hours.

### Example 2

### Preparation of First Fermentation Mixture (2)

100 parts by weight of mugwort and mulberry leaf (1:1, based on weight) was mixed with 100 parts by weight of brown sugar and molasses (5:95) and 2 parts by weight of salt. The mixture was cultured at 45° C. for 48 hours.

### Example 3

### Preparation of First Fermentation Mixture (3)

100 parts by weight of mugwort and green tea leaf (1:9, based on weight) was mixed with 70 parts by weight of molasses and 0.5 part by weight of salt. The mixture was cultured at 60° C. for 24 hours.

### Example 4

### Preparation of First Fermentation Mixture (4)

100 parts by weight of mulberry leaf was mixed with 150 parts by weight of starch syrup and molasses (1:9, based on weight) and 2 parts by weight of salt. The mixture was cultured at 40° C. for 48 hours.

### Example 5

### Preparation of First Fermentation Mixture (5)

100 parts by weight of green tea leaf was mixed with 100 parts by weight of molasses and 2 parts by weight of salt. The mixture was cultured at 50° C. for 30 days.

### Example 6

### Preparation of Second Fermentation Mixture (1)

100 parts by weight of the first organic fertilizer composition prepared in Example 1 was mixed with 100 parts by weight of water, 2.5 parts by weight of glucose and 0.1 part by weight of salt and stirred at 25° C. for 100 minutes. Thus obtained second organic fertilizer composition contained 2×10⁷ Lactobacillus per 1 mL.

### Example 7

### Preparation of Second Fermentation Mixture (2)

100 parts by weight of the first organic fertilizer composition prepared in Example 2 was mixed with 7 parts by weight of brown sugar and 0.5 part by weight of salt and stirred at 30° C. for 80 minutes. Thus obtained second organic fertilizer composition contained 5×10⁸ Lactobacillus per 1 mL.

### Example 8

### Preparation of Second Fermentation Mixture (3)

100 parts by weight of the first organic fertilizer composition prepared in Example 3 was mixed with 7 parts by weight of brown sugar and 3 parts by weight of salt and stirred at 30° C. for 80 minutes. Thus obtained second organic fertilizer composition contained 5×10¹¹ Lactobacillus per 1 mL.

### Example 9

### Preparation of Second Fermentation Mixture (4)

100 parts by weight of the first organic fertilizer composition prepared in Example 3 was mixed with 10 parts by weight of molasses and 0.7 part by weight of salt and stirred at 40° C. for 100 minutes. Thus obtained second organic fertilizer composition contained 3×10¹⁰ Lactobacillus per 1 mL.

### Example 10

### Preparation of Organic Fertilizer Composition (1)

100 parts by weight of the first organic fertilizer composition prepared in Example 1 was mixed with 100 parts by weight of water, 7 parts by weight of glucose and 1 part by weight of salt and stirred at 30° C. for 100 minutes to prepare an organic fertilizer composition A.

Organic fertilizer compositions B, C, D and E were prepared in the same manner, using 100 parts by weight of the first fermentation mixtures prepared in Examples 2, 3, 4 and 5, respectively.

### Example 11

### Preparation of Organic Fertilizer Composition (2)

100 parts by weight of the second fermentation mixture prepared in Example 6 was mixed with 100 parts by weight of water, 5 parts by weight of glucose and starch syrup (1:1) and 1 part by weight of salt and stirred at 40° C. for 100 minutes to prepare an organic fertilizer composition F. Organic fertilizer compositions G, H and I were prepared in the same manner, using 100 parts by weight of the first fermentation mixtures prepared in Examples 7, 8 and 9, respectively.

### Test Example 1

### Preparation of Fertilizer (Liquid Fertilizer)

The organic fertilizer composition F prepared in Example 11 was mixed with pig feces to prepare a fertilizer. 10 wt % of the composition was mixed with 1 ton of pig feces and cultured at room temperature for 1 day.

### Test Example 2

### Cultivation of Red Pepper

Red pepper was planted in soil in May. The organic fertilizer composition G or H prepared in Example 11 was applied once and no plant growth hormone or chemical was used. As a control group, red pepper was cultivated under the same condition except for using commercially available “Utgeoreum fertilizer” (Namhae Chemical) instead of the organic fertilizer composition G or H.

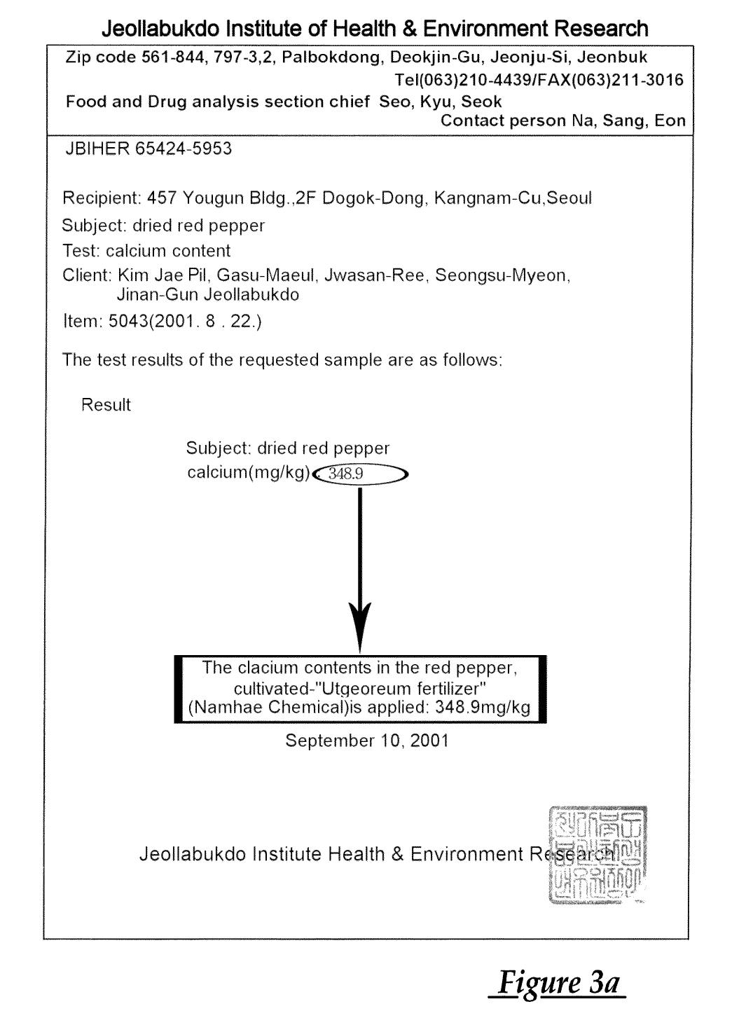

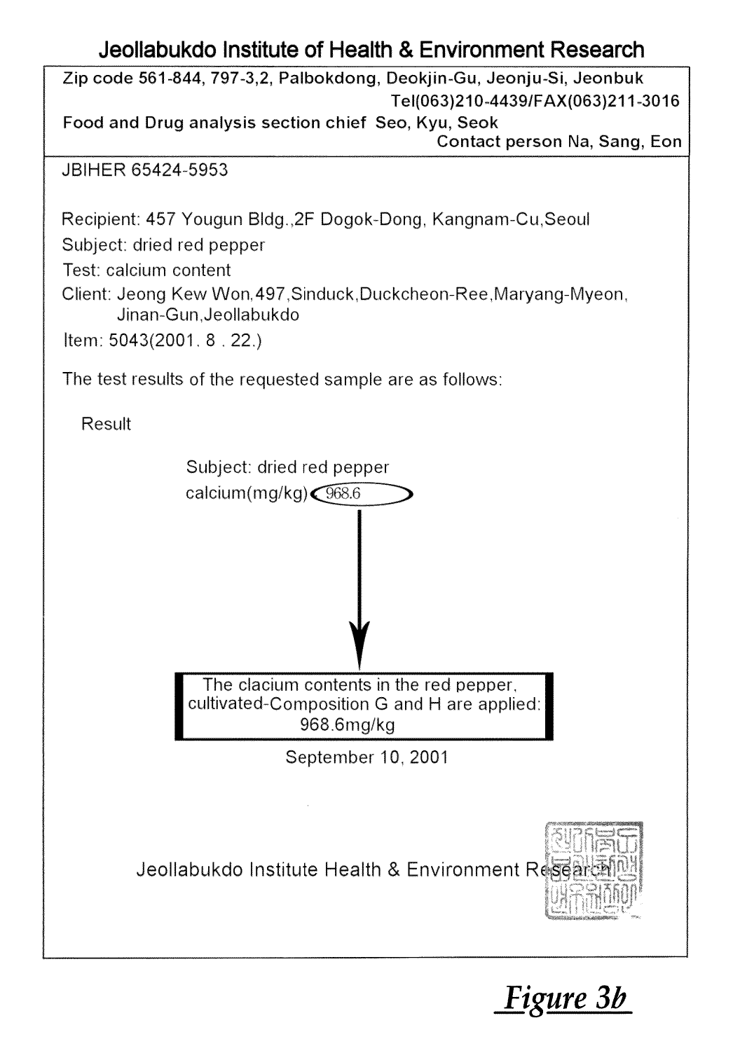

As a result, yield was improved by 20% and 25%, respectively, as compared to the control group and the quality was comparable. As seen from FIG. 3, the calcium content of red pepper increased by more than 270%. Anthrax was observed in some red pepper of the control group.

### Test Example 3

### Cultivation of Rice

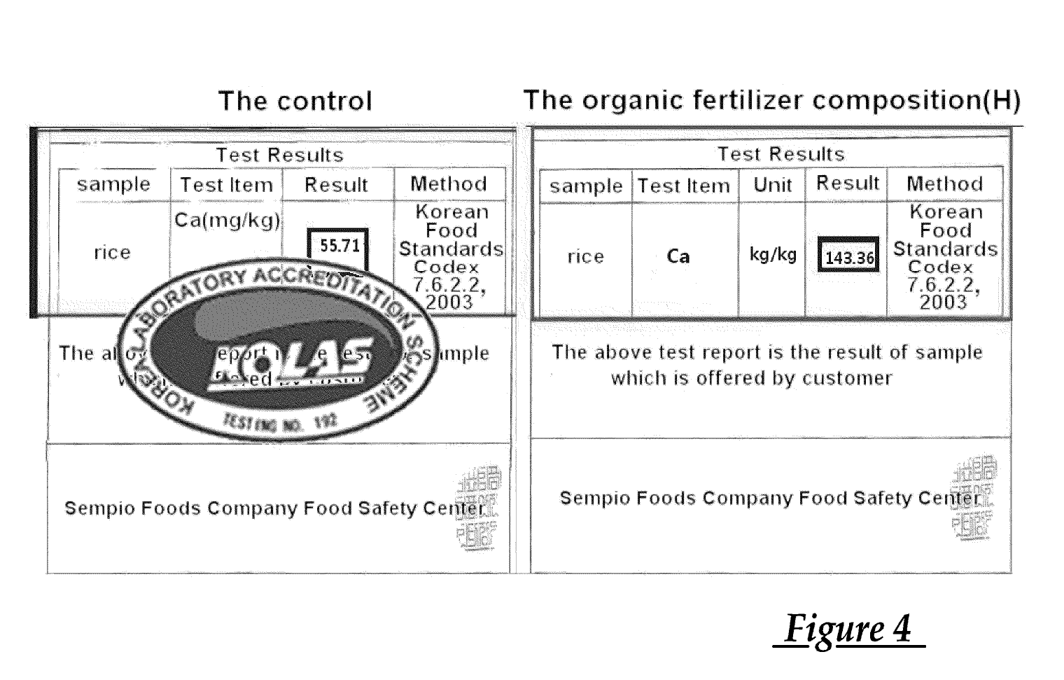

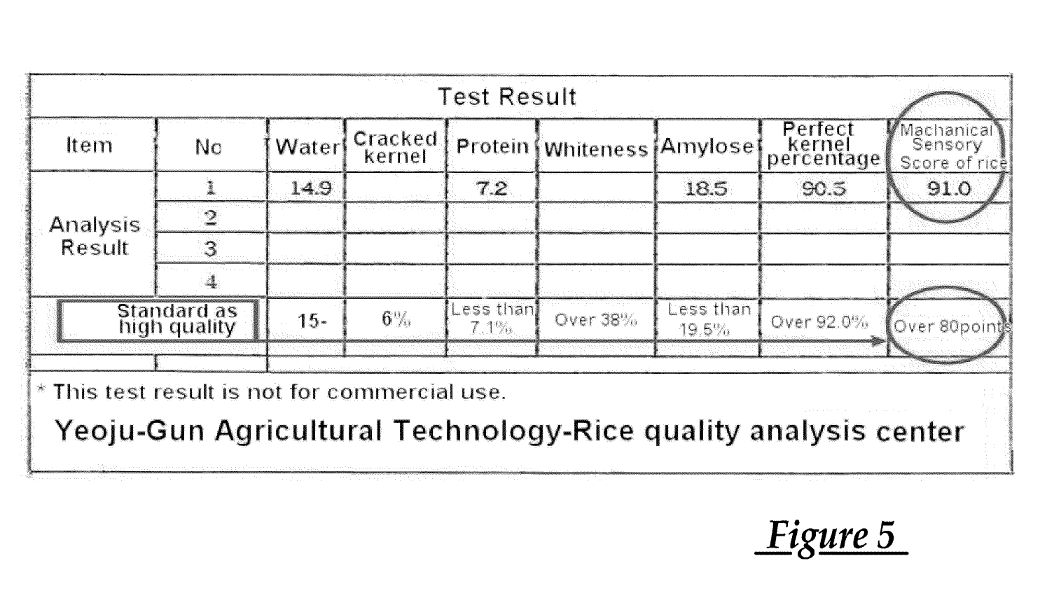

Rice was planted in soil in late February after spraying the organic fertilizer composition H prepared in Example 11. Then, rice was cultivated according to a commonly employed method while reducing the use of agricultural chemicals in half. As a control group, rice was cultivated according to a commonly employed method without using the organic fertilizer composition H but spraying the Utgeoreum fertilizer once. As a result, yield was improved by 15% as compared to the control group. As seen from FIG. 4, the calcium content of rice was about 3 times higher. As seen from FIG. 5, the harvested rice was of high quality. There was no damage from disease or insects at all even though only 50% of agricultural chemicals were used as compared to the control group.

### Test Example 4

### Water Quality Improvement

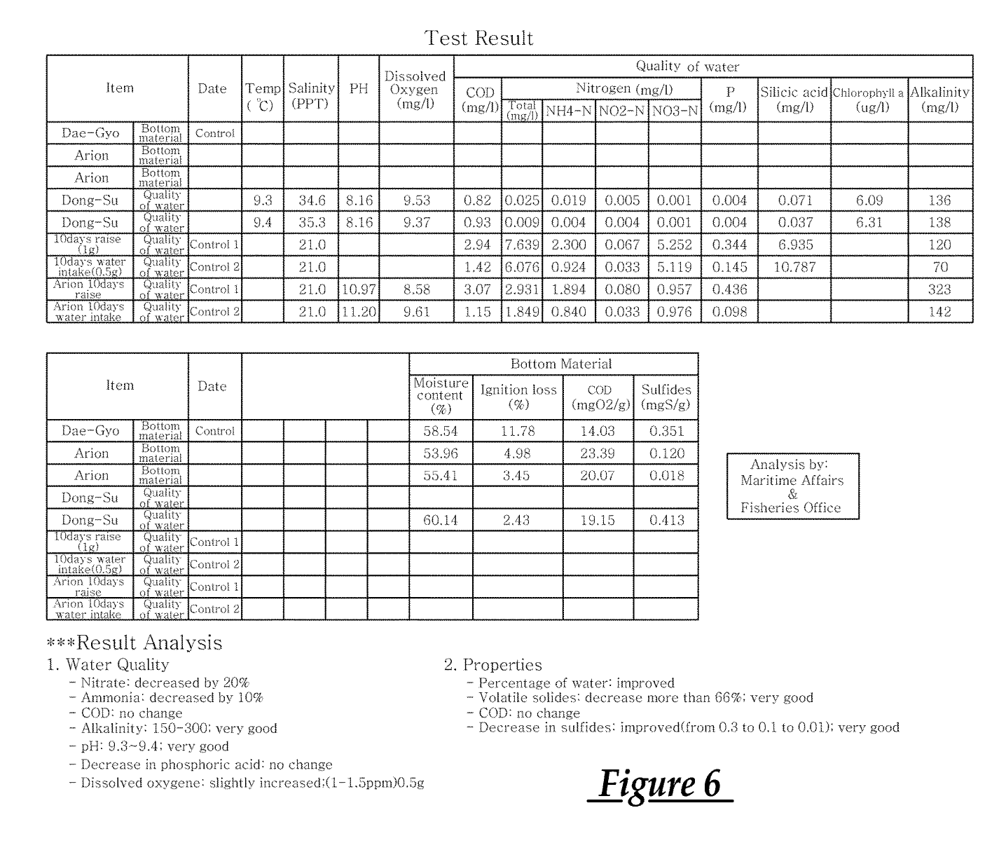

1 L of the organic fertilizer composition F prepared in Example 11 was sprayed onto the water tank of a fish farm per 1 m³. Water quality was analyzed the next day. As seen from FIG. 6, the water quality was improved, with nitrate decreased by 20% and ammonia decreased by 10%.

### Test Example 5

### Water Quality Improvement

Pearl oyster was raised after spraying 1 L of the organic fertilizer composition I prepared in Example 11 per 1 m³. The seawater quality was maintained well and pearls of good quality could be obtained from the pearl oyster.

### Test Example 6

### Fish Farming of Eel

Eel was raised after spraying 1 L of the organic fertilizer composition F prepared in Example 11 per 1 m³. The water quality of the fish farm was maintained well. Significant improvement in water quality was confirmed, with the concentrations of ammonia and acetate remarkably decreased. When the raised eel was cooked about 50 days after the beginning of the test, there was no stench at all not only in the body part but also in the intestines.

### Test Example 7

### Water Quality Improvement

Water flowing out of the drainage channels from (width: 3 m) houses and plants was treated with the organic fertilizer composition C prepared in Example 10 and the organic fertilizer composition F prepared in Example 11. On day 20, water quality was examined. As seen from FIG. 6, the water quality was maintained well and, particularly, the concentrations of ammonia and nitrogen decreased.

### Test Example 8

### Raising of Hen

Egg-laying hen was raised using a livestock feed comprising 5 parts by weight of the organic fertilizer composition A prepared in Example 10 based on 100 parts by weight of assorted feed. One-year-old hens were used for the test. As a control group, egg-laying hen was raised under the same condition except for using assorted feed not containing the organic fertilizer composition A.

As a result, the hens were healthy and more resistant to diseases as compared to the control group. Further, the mineral contents of the eggs lain by the hens were improved by about 70% as compared to the control group. The feather was glossy and the stench of chicken droppings was remarkably decreased.

### Test Example 9

### Raising of Goose

Goose was raised using a livestock feed comprising 3 parts by weight of the organic fertilizer composition F prepared in Example 11 based on 100 parts by weight of assorted feed. 3-month-old geese were used for the test. As a control group, goose was raised under the same condition except for using assorted feed not containing the organic fertilizer composition F. As a result, the geese were healthy and more resistant to diseases as compared to the control group.

As seen from FIG. 6, the meat of the geese raised for 6 months in this manner had a 25% higher mineral content as compared to the control group and the stench of goose feces was decreased.

1. A method for preparing an organic fertilizer composition, comprising:
culturing a first fermentation mixture comprising 100 parts by weight of leaves of one or more plant selected from a grass, a vegetable and a herb, 55-150 parts by weight of one or more sugar selected from glucose, starch syrup, brown sugar and molasses and 0.1-3 parts by weight of salt at 20-60° C. for 24 hours to 1 year;
proliferating a second fermentation mixture comprising 100 parts by weight of the first fermentation mixture, 100 parts by weight of water, 2.5-10 parts by weight of one or more sugar selected from glucose, starch syrup, brown sugar and molasses and 0.1-3 parts by weight of salt at 20-40° C. for 30 minutes to 24 hours; and
mass proliferating a third fermentation mixture comprising 100 parts by weight of the second fermentation mixture, 100 parts by weight of water, 2.5-10 parts by weight of one or more sugar selected from glucose, starch syrup, brown sugar and molasses and 0.1-3 parts by weight of salt at 20-40° C. for 1-12 hours.

2. A method for preparing an organic fertilizer composition, comprising:
culturing a first fermentation mixture comprising 100 parts by weight of leaves of one or more plant selected from a grass, a vegetable and a herb, 55-150 parts by weight of one or more sugar selected from glucose, starch syrup, brown sugar and molasses and 0.1-3 parts by weight of salt at 20-60° C. for 24 hours to 1 year; and
proliferating a second fermentation mixture comprising 100 parts by weight of the first fermentation mixture, 100 parts by weight of water, 2.5-10 parts by weight of one or more sugar selected from glucose, starch syrup, brown sugar and molasses and 0.1-3 parts by weight of salt at 20-40° C. for 30 minutes to 24 hours.

3. An organic fertilizer composition made from the method of claim 2.

4. A fertilizer including the organic fertilizer composition of claim 3.

5. A fertilizer which is made from the steps of:
mixing the organic fertilizer composition of claim 3 with one or more selected from livestock excretions and food waste, and
fermenting the mixture at the temperature ranging 10 to 45° C. in the time ranging 1 minute to five days.

6. A soil conditioner including the organic fertilizer composition of claim 3.

7. A water quality improving agent including the organic fertilizer composition of claim 3.

8. A livestock feed additive including the organic fertilizer composition of claim 3.

9. A stench eliminator including the organic fertilizer composition of claim 3.

10. An organic fertilizer composition made from the method of claim 1.

11. A fertilizer including the organic fertilizer composition of claim 10.

12. A fertilizer which is made from the steps of:
mixing the organic fertilizer composition of claim 10 with one or more selected from livestock excretions and food waste, and
fermenting the mixture at the temperature ranging 10 to 45° C. in the time ranging 1 minute to five days.

13. A soil conditioner including the organic fertilizer composition of claim 10.

14. A water quality improving agent including the organic fertilizer composition of claim 10.

15. A livestock feed additive including the organic fertilizer composition of claim 10.

16. A stench eliminator including the organic fertilizer composition of claim 10.
